(19) United States
(12) Patent Application Publication
(10) Pub. No.: US 2018/0221054 A1
(43) Pub. Date: Aug. 9, 2018

(54) SURGICAL DEVICE AND METHOD OF USE

(71) Applicant: Corinth MedTech, Inc., Cupertino, CA (US)

(72) Inventor: Csaba Truckai, Saratoga, CA (US)

(73) Assignee: Corinth MedTech, Inc., Cupertino, CA (US)

(21) Appl. No.: 15/884,212

(22) Filed: Jan. 30, 2018

Related U.S. Application Data
(60) Provisional application No. 62/456,534, filed on Feb. 8, 2017.

(51) Int. Cl. A61B 17/34; A61B 18/14; A61B 17/00

(52) U.S. Cl. CPC A61B 17/3468; A61B 2018/00577; A61B 17/00234; A61B 18/14

(57) ABSTRACT

A tissue resection system includes a introducer sleeve with a hub. An obturator having a shaft with a blunt tip is adapted for insertion through the introducer sleeve, and an electrosurgical resecting device with an elongated extension member is adapted for insertion through the introducer sleeve.

### CROSS-REFERENCE TO RELATED APPLICATION

This application claims the benefit of Provisional Patent Application No. 62/456,534 (Attorney Docket No. 42005-710.101), filed on Feb. 8, 2017, the full disclosure of which is incorporated herein by reference.

### BACKGROUND OF THE INVENTION

### 1. Field of the Invention

The present invention relates generally to devices and methods for resecting and removing tissue from the interior of a patient's body. More particularly, the present invention relates to transurethral resection of prostate tissue to treat benign prostatic hyperplasia.

Electrosurgical cutting devices often comprise a shaft or sleeve having a tissue extraction lumen with one or more radio frequency (RF) cutting blades arranged to resect tissue which may then be drawn into the extraction lumen, often via vacuum assistance through a cutting window. Most such electrosurgical tissue cutting devices rely on manually engaging the cutting window against the target tissue to be resected.

For resection of remote tissue sites, such as the prostate, it is usually desirable to introduce the surgical cutter through a tubular introducer device. While such tubular introducers can be advanced “blind,” i.e. without direct optical visualization, it is frequently desirable to prove such direct visualization. For example, it would be desirable to use an endoscope to observe the urethra while transurethrally advancing an introducer sheath for subsequent resection of the prostrate. Once the introducer sheath is in place, however, it will be necessary to advance the cutter through the introducer sheath which can require that fluid recirculation pumps be disconnected from the introducer sheath and reconnected to the surgical cutter. Such an exchange can be time consuming, and often the surgical cutter will require a different pumps and/or or pump interface than does the introducer sheath.

For these reasons, it would be desirable to provide systems of urethral and other introducers which are particularly adapted to allow rapid connection and deployments of the both the visualization and resection components of the system. It would be particularly desirable if the introducer sheaths were easily connectable to and disconnectable from a pumping and control system that can be used both for initial advancement of the introducer sheath and for subsequent connection of the surgical cutter or other surgical tool. At least some of these objectives will be met by the inventions described below.

### 2. Description of the Background Art

Related patents and published applications include U.S. Pat. No. 8,221,404; U.S. Pat. No. 7,744,595; U.S. Pat. Publ. 2014/0336643; U.S. Pat. Publ. 2010/0305565; U.S. Pat. Publ. 2007/0213704; U.S. Pat. Publ. 2009/0270849; and U.S. Pat. Publ. 2013/0090642. See also commonly assigned, published applications: US 2014/0336643; US 2017/0105748; US 2017/0105607; US 2017/0333120; and US 2017/0333119.

### SUMMARY OF THE INVENTIONS

In a first aspect, the present invention provides a tissue resection system including an introducer sleeve having a distal end and a hub at a proximal end. An obturator has a shaft with a blunt tip, and the shaft and the blunt tip are adapted for removable insertion through a central passage of the introducer sleeve. The system further includes an electrosurgical resecting device having a shaft or other elongated extension member also adapted for removable insertion through the central passage of the introducer sleeve. In particular, the obturator and the resecting device are configured to be exchanged within the introducer sleeve while the sleeve is present in the urethra of a patient.

In specific embodiments of the system, a controller console including at least a radiofrequency (RF) energy source, a fluid perfusion source, a fluid aspiration source, and a pressure sensor is provided for interconnection with both the obturator and the resection device, preferably using a single (common) umbilical connector that can be connected and disconnected as a procedure progresses.

Usually, the obturator is configured to detachably connect to at least the fluid perfusion source, a fluid aspiration source, and a pressure sensor of the controller console, but not to the RF energy source. Usually, the electrosurgical resecting device is configured to detachably connect to all of the fluid perfusion source, the fluid aspiration source, the pressure sensor, and the RF energy source of the controller console.

In other embodiments, the system further comprises an umbilical cable including at least tubes for connecting to the fluid perfusion source, the fluid aspiration source, and the pressure sensor. Often, the umbilical further comprises an RF cable for connecting to the RF source. Alternatively, the system may further comprise an RF cable for connecting to the RF source, wherein the RF cable is separate from the umbilical.

In still further embodiments of the system, the obturator shaft may have first and second flow channels for fluidic communication with the fluid perfusion source and the fluid aspiration source, respectively. Additionally, the extension member of the resecting device may have first and second flow channels for fluidic communication with the fluid perfusion source and the fluid aspiration source respectively, and the obturator shaft further has an endoscope-receiving passageway extending therethrough. In some instances, the extension member of the resecting device further may have an endoscope-receiving passageway extending therethrough, and in other instances, the obturator shaft further may have a third channel therethrough adapted for fluidic communication with a pressure sensor and/or the resecting device further may have a third channel therethrough adapted for fluidic communication with a pressure sensor.

In a second aspect, the present invention provides a method for treating a patient's prostate. An obturator is connected to a controller console having a fluid perfusion source. A sleeve carrying the obturator and an endoscope is advanced though the patient's urethra while introducing a fluid from the controller console though the obturator into the urethra. The urethra is observed a through the endoscope while advancing the sleeve and introducing the fluid, and sleeve advancement is ceased after a user observes that a distal end of the sleeve has reached the prostate. The obturator is then disconnected from the controller console, and the endoscope is removed from the obturator. The obturator is then exchanged for a resection device within the sleeve. The resection device is then connected to the fluid source and to a radiofrequency (RF) energy source both of which are within the controller console. The same endoscope is placed in the resection device, and the prostate is resected using the resection device with RF energy from the RF energy source while circulating fluid from the controller console in the urethra and observing the resection with the endoscope.

In specific embodiments of the methods, the controller console further includes at least a fluid aspiration source, and the method further comprises aspirating fluid from the urethra using the aspiration source while advancing the obturator and/or resecting the prostate. Additionally, the controller console may further include at least a pressure sensor, and the method may further comprise measuring pressure in the urethra using the pressure sensor while advancing the obturator and/or resecting the prostate. In other instances, the fluid perfusion source, the fluid aspiration source, and the pressure sensor may be interchangeably connected from the controller console to both the obturator and the resection device using a single umbilical cord. In alternative instances, the RF energy source may be individually connected from the controller console to the resection device using a power cord separate from the umbilical cord.

### BRIEF DESCRIPTION OF DRAWINGS

FIG. 1 is a view of a tissue resecting device and a block diagram of systems and operating components corresponding to the invention.

FIG. 2 is a perspective view of an overall tissue resection system that includes the resecting device of FIG. 1 as well as an obturator device and an introducer device that are assembled in different configurations for different parts of a surgical procedure.

FIG. 3 is a perspective view of the obturator device of FIG. 2 inserted through the introducer device of FIG. 2 from an angle that shows components and features of the device.

FIG. 4 is an enlarged perspective view of the distal end of the obturator device and introducer sleeve of FIG. 3.

FIG. 5 is a perspective view of the resecting device of FIG. 2 assembled with the introducer device of FIG. 2.

FIG. 6 is an enlarged perspective view of the working end of the resecting device and introducer sleeve of FIG. 5.

FIG. 7 is an enlarged perspective view of the working end of the resecting device of FIG. 2 without the introducer sleeve.

FIG. 8A is a perspective view of the working end of the resecting device and introducer sleeve of FIG. 5 from different angle shows the working end of the resecting device during reciprocation.

FIG. 8B is a perspective view similar to that of FIG. 8A showing the stroke axial of the working end of the resecting device relative to the introducer sleeve and endoscope.

FIG. 9 is a schematic view of the controller console, fluid management system and footswitches of the invention.

FIG. 10A is a schematic view of a method of the invention wherein an endoscope is inserted into the obturator and introducer assembly and fluid flow tubing is coupled to the obturator for providing irrigation, wherein the assembly is shown being introduced through a patient's urethra to access the prostate under endoscopic vision and continuous flow irrigation.

FIG. 10B illustrates a subsequent step of the method wherein the obturator is withdrawn from the introducer device leaving the introducer sleeve in place for access to the patient's prostate.

FIG. 10C illustrates a further subsequent step wherein the endoscope is inserted into the resecting device, and flow tubing is coupled to the resecting device, after which the resecting device is inserted through the introducer sleeve remaining in the patient to access the treatment site in the prostate or bladder.

FIG. 11 consists of two views of the working end of the resecting device in different orientations; in one orientation, the window and electrode are facing downward and in the second orientation the window in the electrode are facing in an upward direction.

FIG. 12 shows a perspective view of another variation of resecting device in which the central core carries the endoscope in the working end is rotatable within a stationary handle that can be actuated to reciprocate the working end.

### DETAILED DESCRIPTION OF THE INVENTION

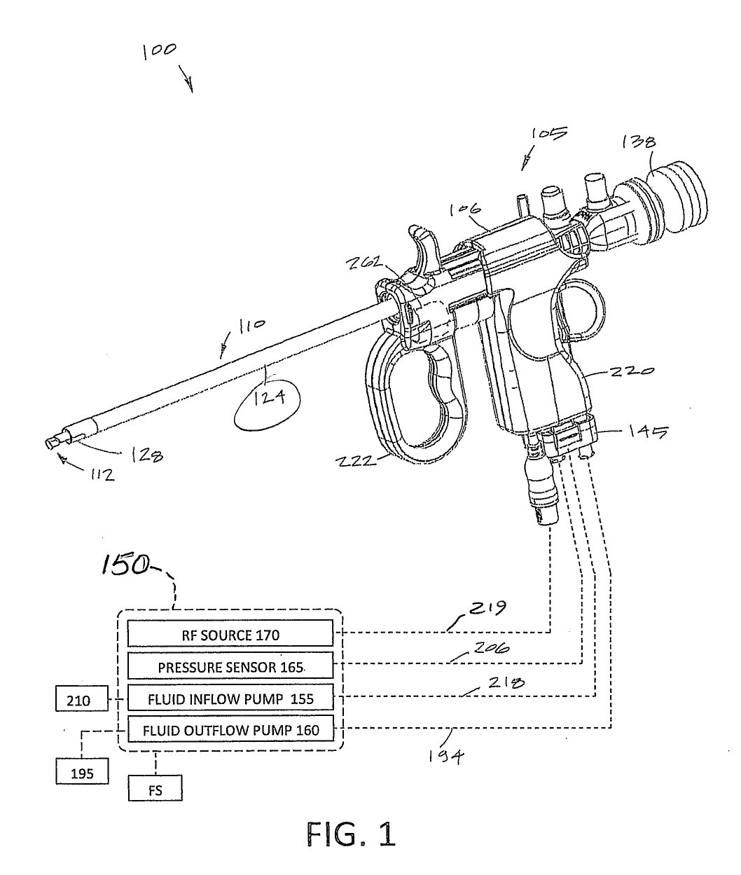

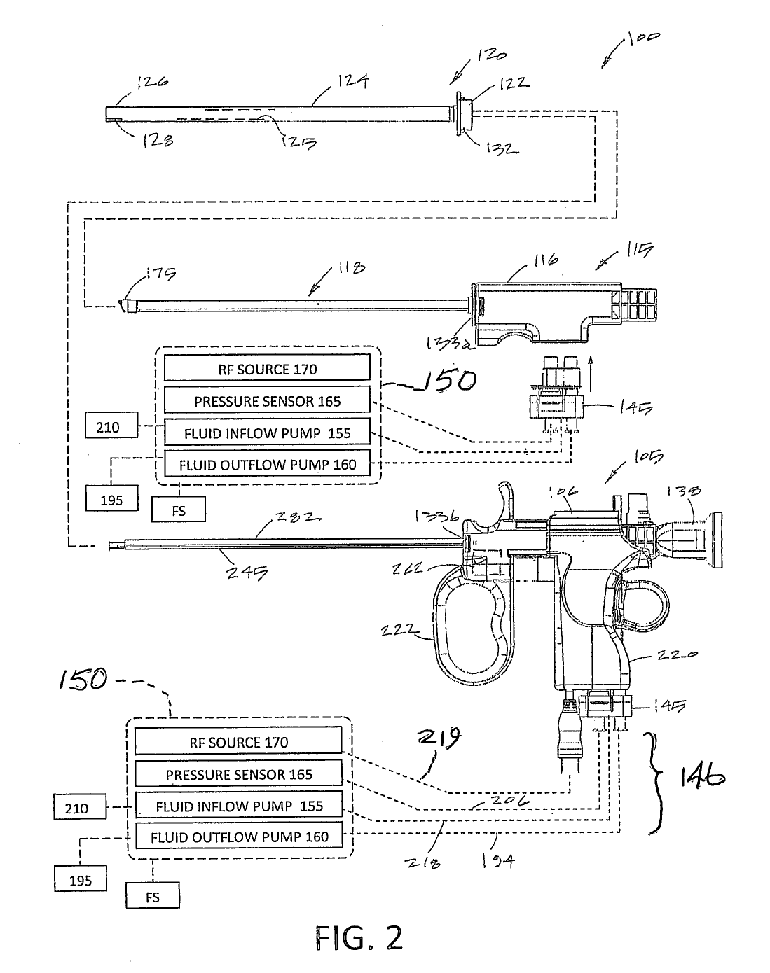

FIGS. 1 and 2 illustrate an electrosurgical tissue resecting system 100 for use in urological procedures to resect tissue, for example in a procedure to resect tissue to treat benign prostatic hyperplasia (BPH). FIG. 1 shows a resecting device or probe 105 as an assembly described below for resecting tissue wherein the device 105 has a handle 106 coupled to an elongated shaft or extension portion 110 that carries an electrosurgical working end 112.

FIG. 2 is an exploded view of the system 100 that includes three devices that are used in combination for transurethral insertion and thereafter for tissue resection. In the lower part of FIG. 2, the resecting device 105 of FIG. 1 is shown. In the middle part of FIG. 2, an obturator device 115 with handle 116 and elongate obturator shaft 118 is shown. In the upper part of FIG. 2, an introducer device 120 which has proximal body or hub 122 coupled to elongate thin wall sleeve 124.

As will be described in detail below, the obturator 115 in combination with introducer device 120 of FIG. 2 is used in a first step of a method of the invention for transurethral introduction of the assembly into the patient's prostate. After introducing the obturator shaft 118 and introducer sleeve 124 into the patient, the operator removes the obturator 115 leaving the introducer device 120 in the patient. Thereafter, the physician inserts the resection device 105 through the introducer sleeve 124 to access the working space.

Now describing the introducer device 120 in more detail, referring to FIG. 2, the introducer sleeve 124 can comprise a thin-wall metal, plastic tube, or a combination thereof that defines a lumen 125 therein. The introducer sleeve 124 can have a diameter or oval shape with a mean diameter ranging from 6 mm to 12 mm. The distal end 126 of the sleeve 124 as an electrically nonconductive tip portion 128 which will be described in more detail below. The hub 122 of the introducer device 120 has a latch mechanism as is known in the art for locking the hub 122 into either the obturator device 115 or the resecting device 105. In one variation, the latch mechanism comprised at least spring tab 132 that can lock into at least one receiving opening 133a in the obturator device 115 or opening 133b in the resecting device 105. The latch mechanism is configured to lock the introducer sleeve 124 in a selected rotational orientation relative to the resecting device 105.

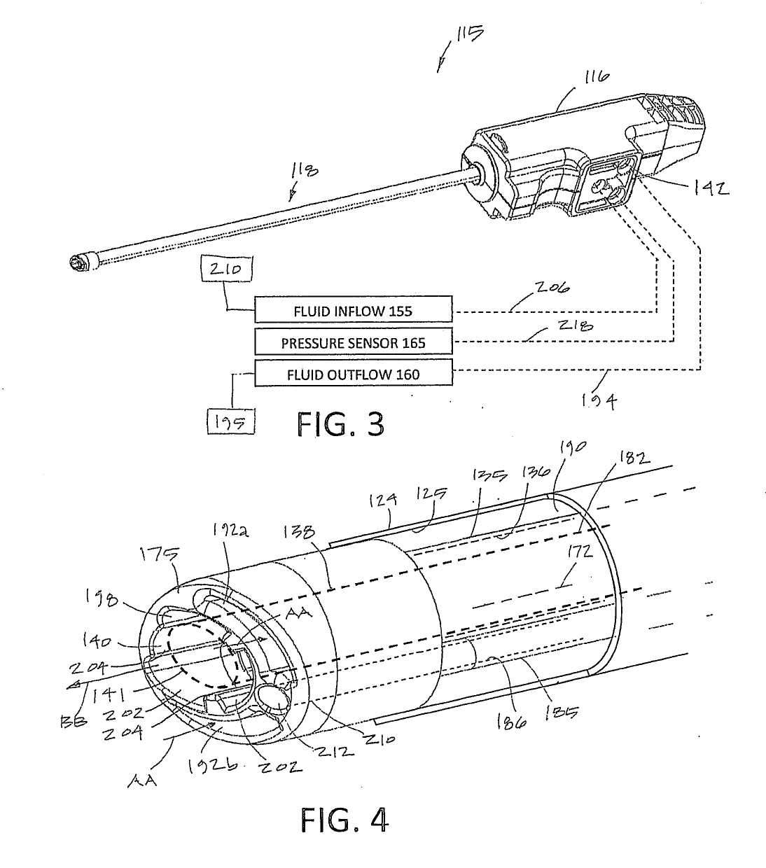

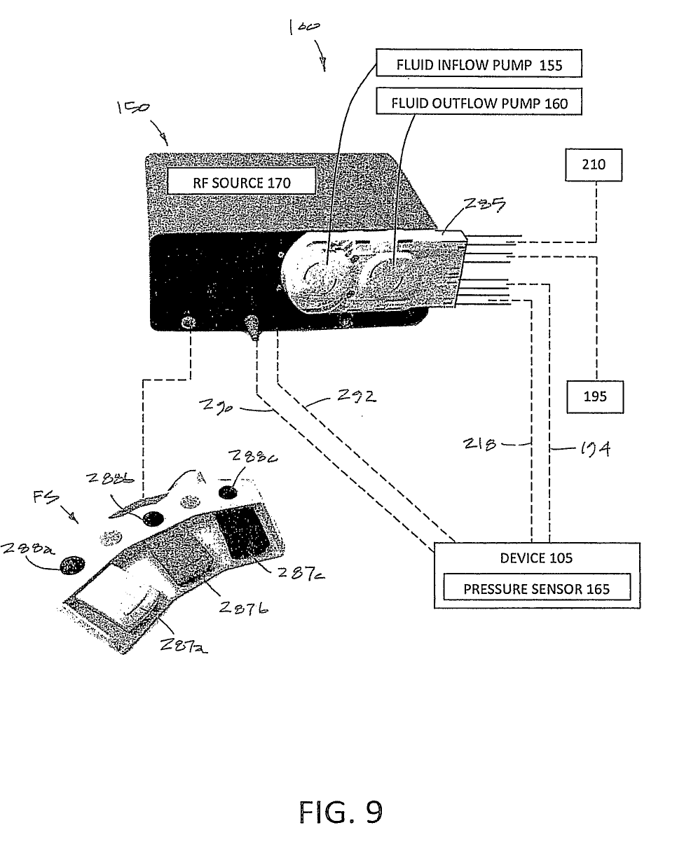

Referring now to FIGS. 2, 3 and 4, the obturator device 115 is shown in more detail. Of particular interest, the obturator's elongate shaft 118 includes a obturator sleeve or first sleeve 135 that has an interior channel 136 for receiving an endoscope 138 (FIG. 2) that allows for direct vision into the patient's urethra during introduction of the assembly of the obturator 115 and the introducer device 120. In FIG. 4, a distal opening 140 in the obturator communicates with the interior channel 136 in the first sleeve 135. It can be understood that the distal end 141 of endoscope 138 can be positioned in said open termination 140 to provide for endoscopic viewing. The endoscope can be adapted with a straight viewing angle or more often with an oblique viewing angle as shown in FIG. 4. The obturator handle 116 can comprise a molded plastic assembly that includes a fitting 142 for locking the endoscope 135 in the handle 140. FIGS. 2 and 3 further show that the obturator handle 140 includes a female fitting 144 that is adapted to receive connector 145 (FIG. 2) that couples fluid flow tubing to a controller console 150 and inflow and outflow pumps 155 and 160. In FIGS. 2 and 9, it can be understood that the controller console 150 and inflow pump 155 and outflow pump 160 are configured to provide a circulating irrigation flow within the urethra during insertion of the introducer sleeve 120 and obturator 115 as well as for irrigation the working space during the subsequent resection procedure. The controller console 150 further carries a pressure sensor 165 and an RF source 170, both of which will be described in more detail below.

FIG. 4 shows that the obturator shaft 118 extends along axis 172 to a rounded or blunt distal tip or nose 175 that can be fabricated of any type of suitable plastic or metal, and in one variation is a transparent plastic. Still referring to FIG. 4, the thin wall first sleeve 135 with bore 136 therein accommodates the endoscope shaft 182 (see FIG. 2). The thin wall first sleeve 135 can have a diameter ranging from 5 mm to 10 mm. The obturator shaft 118 further includes a smaller diameter thin wall second sleeve 185 with an interior channel 186 that extends into obturator handle 116. The diameter of interior channel 186 can range from 0.5 mm to about 2.5 mm and is adapted to communicate with pressure sensor 165 in the controller console 150 for purposes described further below.

Referring to FIG. 4, it can be seen that a first flow channel 190 in the assembly of the obturator 115 and introducer sleeve 124 is defined by the space between exterior of the obturator shaft 118 (i.e., sleeves 135 and 185) and wall of lumen 125 of introducer sleeve 124. The first flow channel 190 is used for fluid outflows in communicates in the distal direction with first and second open terminations 192a and 192b in the nose 175 of the obturator 115, with the flows indicated by arrows AA. The flow channel 190 extends in the proximal direction through handle 116 and fitting 144 to flexible tubing 194 (FIG. 3) that extends to the outflow pump 160 which can be a peristaltic pump or other similar pump. Ultimately, the flexible tubing 194 and fluid outflows are directed to a fluid collection reservoir 195 (FIGS. 2 and 3).

Referring again to FIG. 4, a second flow channel 198 in the assembly of the obturator 115 and introducer device 120 is defined by the space between the outer surface of the endoscope shaft 182 and the inner wall of lumen 136 of obturator sleeve 135. As can be seen in FIG. 4, the distal nose 175 of the obturator has axial grooves 202 and edges 204 in the open termination 140 of the nose 175 which support the endoscope shaft 182. Thus, a fluid inflow in the distal direction through space or channel 198 can exit the distal nose 175 to irrigate the urethra, with the flow indicated by arrows BB. The second flow channel 198 extends in the proximal direction to handle 116 and fitting 144 and thereafter to flexible tubing 206 which extends to the inflow pump 155 and a fluid source 210 which can be a saline bag.

In addition to the flexible tubes 194, 206 and 218, an RF cable 219 will usually be provided to selectively interconnect the controller console 150 to the obturator 115 and to the resecting device or other probe 105. In particular, when connected to the obturator 115, the controller console 150 can deliver (infuse) saline or other fluids and collect (aspirate) saline or other fluids using pumps 155 and 160 and pressure sensor 165. When connected to the resecting device or other probe 105, the controller console 150 can deliver RF ablation or other electrosurgical or electrocautery current to the working end 112, as well as infusing and aspirating fluids and measuring pressure. Usually, the flexible tubes 194, 206 and 218 will be combined in a single umbilical cord 146 having the connector which can connect to both the obturator handle 116 and to a handle portion or grip 220 of the resecting device or other probe 105. An RF cable 219 can be separate or combined with the tubes into a single umbilical cord (FIGS. 2 and 3).

Still referring to FIG. 4, the third flow channel 210 extends from open port 212 in nose 175 that communicates with interior channel 186 in second sleeve 185 of the obturator device 115. This third flow channel 210 comprises a pressure sensing channel and extends though obturator handle 116 and fitting 144 to flexible tubing 218 that communicates with the pressure sensor 165 in the controller console 150. The method of using the pressure sensing system will be described further below.

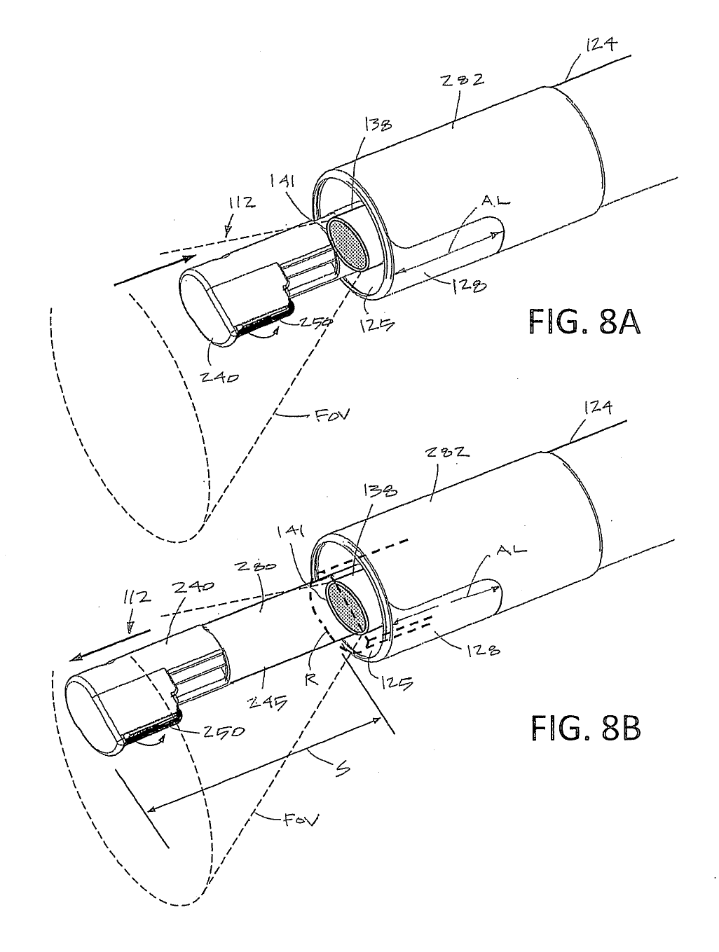

Now turning to the resecting device 105 as shown in FIGS. 2, 5 and 6, the device 105 has a handle 106 coupled to shaft or extension portion 110 that has multiple sleeves, all of which can be introduced through the lumen 125 of introducer sleeve 124 as shown in FIG. 1. The handle 106 comprises a proximal handle portion or grip 220 and a movable distal handle portion or grip 222 that is movable between an extended position and a retracted position. The physician can squeeze and release the proximal and distal grips 220 and 222 to reciprocate the electrosurgical working end 112 relative to the distal end 141 of the endoscope 138 between ‘extended’ and ‘retracted’ positions as will be described below. The electrosurgical working end 112 can have a stroke S as shown in FIGS. 6 and 8B which can range from 5 mm to 20 mm, and more often between 8 mm and 15 mm.

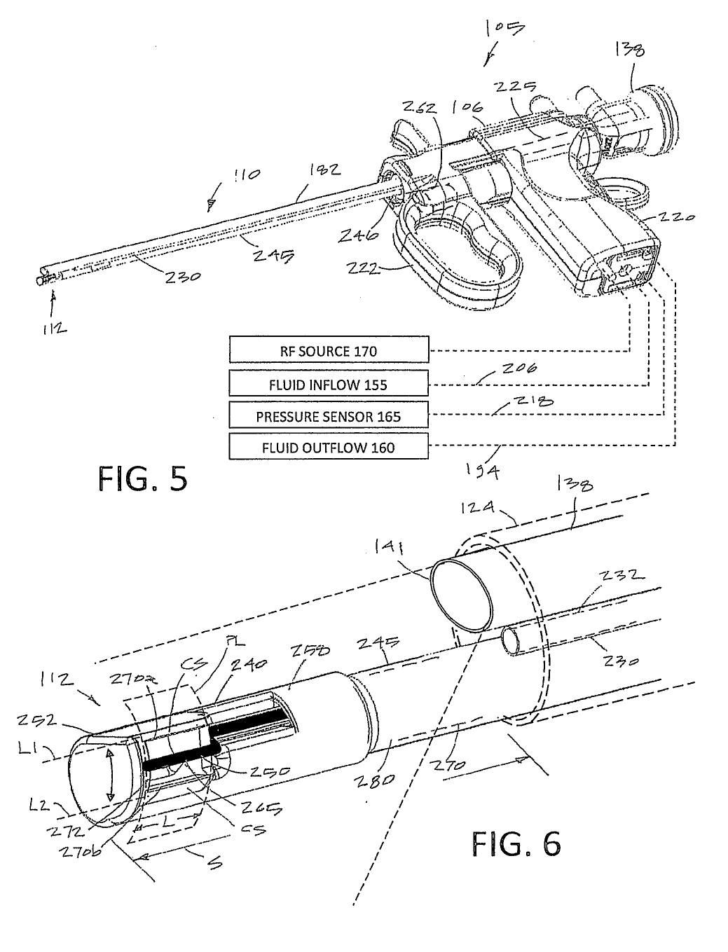

In FIGS. 5 and 6, it can be seen that the extension portion 110 of the resecting device 105 includes endoscope sleeve 225 in handle 106 that accommodates the shaft 182 of endoscope 138. FIGS. 5 and 6 further show that a sensor sleeve 230 is coupled to handle 106 which is similar to sleeve 185 in the obturator device 115 wherein the lumen 232 in sleeve 230 communicates through tubing 218 with the pressure sensor 165 in controller console 150.

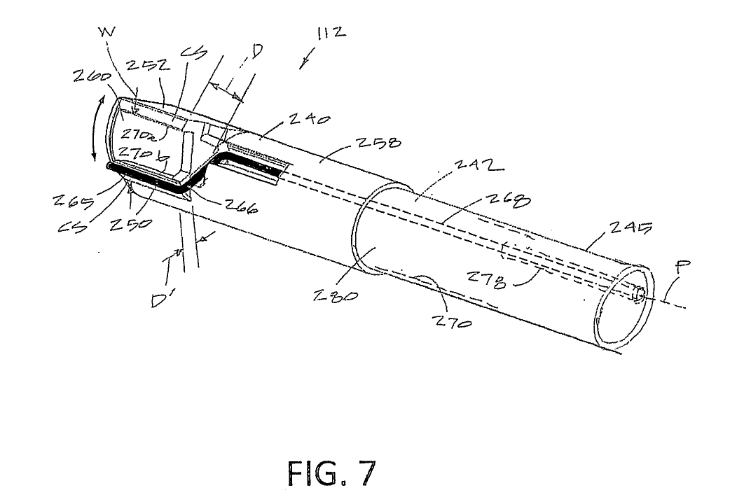

Referring to FIGS. 6 and 7, the working end 112 of the resecting device 105 comprises a ceramic housing 240 that is coupled to the distal end 242 of reciprocating sleeve 245. Thus, it can be understood that the proximal end 246 of reciprocating sleeve 245 is coupled to the distal grip 222 (FIG. 5) while the endoscope sleeve 225 and endoscope 138 are coupled to the stationary proximal grip 220. The proximal and distal grips 220 and 222 of handle 106 thus can be squeezed and released to axially reciprocate the working end 112 relative to endoscope sleeve 225 and endoscope 138. In a variation, a spring in the handle 106 urges the proximal and distal grips 220 and 222 to the spaced apart position which corresponds to the ‘extended’ position of working end 112.

In the variation of FIGS. 6 and 7, the asymmetric or offset housing 240 can comprise a ceramic material such as zirconium oxide, aluminum oxide or another similar material as is known in the art. In FIGS. 6, 7 and 8A, it can be seen that the ceramic housing 240 has a ceramic surface CS that carries window 260 that is offset from the cylindrical surface 258 on each side by a predetermined offset dimensions D and D′, when measured radially outward from a tangent to the shaft cylindrical surface 258. Thus, the electrode 250 is configured to moves in an arc that has a radius extending to a point P adjacent the inner wall of the reciprocating sleeve 245 (FIG. 7). In a variation, the ceramic surface offset dimension D can range from 2 mm to 8 mm, and the offset dimension D′ can range from 1 mm to 4 mm.

As can be further be seen in FIGS. 6 and 7, the ceramic surface CS extends around sides of the window and has an arc shape to allow the longitudinal portion 265 of electrode 250 to sweep past the lateral window edges 270a and 270b and distal edge 272 to shear tissue. In general, the window has a rectangular shape with a length L ranging from 5 mm to 15 mm and a width W ranging from 2 mm to 8 mm. In general, the window 260 in ceramic housing 240 can be configured to have a radial arc relative to the electrode shaft 268 ranging between 30° and 180°.

Referring to FIGS. 6-7, one variation of resecting device 105 has an electrode 250 that can be a tungsten or stainless steel wire wherein electrode portion 265 is adapted to sweep across window 260 at any suitable rate, for example from 1 Hz to 500 Hz.

As can be understood from FIGS. 6 and 7, the electrode portion 265 moves back and forth akin to a windshield wiper across window 260 in the ceramic housing 240. A number of mechanisms can be used to effectuate the desired movements of the electrode 250, or the motor drive unit 262 simply can be controlled by software to move in intermittent clockwise and counter-clockwise directions. In one variation, the elongated shaft portion 268 of the electrode 250 includes a twist-resistant hypotube 278 over the shaft portion 268 to prevent twisting under loads (FIG. 7). Thus, the distal section 265 of the electrode 250 will able to move back and forth entirely across window 260 to cut tissue without twisting of the electrode shaft 268 even when the engaged tissue offers resistance to movement of electrode portion 265. Referring to FIGS. 6 and 7, the outer surface of reciprocating sleeve 245 can comprise the return electrode 280.

FIGS. 8A-8B illustrate another aspect of the invention wherein the working end 112 of resecting device 105 provides for particular angular orientation between the ceramic housing 240/electrode 250 and the distal tip 141 of endoscope 138. As can be seen in FIGS. 8A-8B, the endoscope 138 as an angled field of view, which may be from 5° to 20°. The ceramic housing 240 and electrode 250 carried by the end of shaft 245 are adapted to reciprocate in stroke S within the endoscope's field of view FOV. Further, the window 260 has a particular angular orientation relative to the axis of endoscope 138 wherein the offset portion 252 of housing 240 and electrode 250 are always positioned in the field of view FOV throughout the medial and distal portion of the stroke S. Stated another way, referring to FIG. 6, the plane PL in which the electrode 250 moves between it oscillation limits L1 and L2 is always in the field of view FOV.

FIG. 8B shows a further aspect of the invention wherein the ceramic body 240 and the electrode 250 in a fully retracted position indicates at R can be pulled into bore 125 of sleeve 124. For this reason, the distal tip 282 of sleeve 124 has a thin dielectric member 128 which extends radially around the tip 282. The dielectric member 128 has an angular dimension that is greater than the distance between limit L1 limit L2 of the electrode movement as shown in FIG. 6. The dielectric member 128 has an axial length AL that is determined by the dimension in which the ceramic member 240 and electrode 250 can be retracted into the bore 125 at the distal end of sleeve 124. It can be understood that the purpose of the dielectric member 128 is to ensure the electrode 250 is not in close proximity to any conductive material of the sleeve 124 which serves as a return electrode 280.

FIG. 9 illustrates a controller 150 of the invention which was shown in a block diagram in FIGS. 1 and 2. The controller console carries that RF source 170 and motors that drive of fluid inflow peristaltic pump 155 and the fluid outflow peristaltic pump 160. In one variation shown in FIG. 9, a cassette 285 carries inflow tubing 218 that extends from the fluid source 210 to the resecting device 105 and outflow tubing 194 that extends from the resecting device 105 through the cassette to the collection reservoir 195. Further, foot switch assembly FS is coupled to the controller console 150 and includes 3 foot pedals 287a, 287b and 287c. The foot switch assembly further carries depressible buttons 288a, 288b, and 288c. In one variation the first pedal 287a is used to activate the “cut mode” wherein an ablation waveform of RF current is delivered to the resecting device 105 and electrode 250 to cut tissue. The second pedal 287b is used to activate the “coagulation mode” wherein the coagulation waveform is delivered to electrode 250. The third pedal 287c is adapted to provide a high flow rate of fluid for flushing the working space, for example when blood is present and the physician wishes to clear the site. Such interrogation mode can deliver fluid flows ranging between 400 mL per minute and 800 mL per minute.

Referring to FIG. 9, the controller 150 includes algorithms to control or maintain fluid pressure at a pre-selected level in a working space in response to signals from pressure sensor 165. In one variation, the first button 288a is adapted to lower the set pressure in the working space by a predetermined amount for example 1 to 5 mm Hg. Similarly, the second button 288b is adapted to increase a set pressure in the working space by a predetermined amount. For example, the controller 150 may have a preset to maintain the fluid pressure in the working space of 50 mmHg. If the physician then wants to increase or decrease the fluid pressure in the working space, he or she simply can press either button 288a or 288b to adjust the fluid pressure in the working space. In another variation, the third button 288c can be used to provide a reverse flow through the system and outflow channel 270 in the resecting device 105. At times, it is possible for tissue chips to clog the outflow channel. A reverse flow may be use to push the tissue chips distally out through window 260 to eliminate the clog. In one variation, the actuation of such a reverse flow with the device still in the working space reverses both pumps 155 and 160 and control algorithm can still maintain the set pressure in the working space. In another variation, the resecting device can be withdrawn from the working space and the reverse flow can be actuated only on the outflow pump 160 to eject the tissue clog into a waste container. The resecting device and introducer can have a suitable sensor such as a contact switch, magnetic switch or the like that engages the introducer 120 to signal the controller 150 whether the resecting device in the introducer or not deployed to determine whether to reverse flows in both pumps 155, 160, or only the outflow pump 160 for purposes just described.

Still referring to FIG. 9, an RF cable 290 and pressure sensor cable 292 also extend from the controller 150 to the resecting device 105.

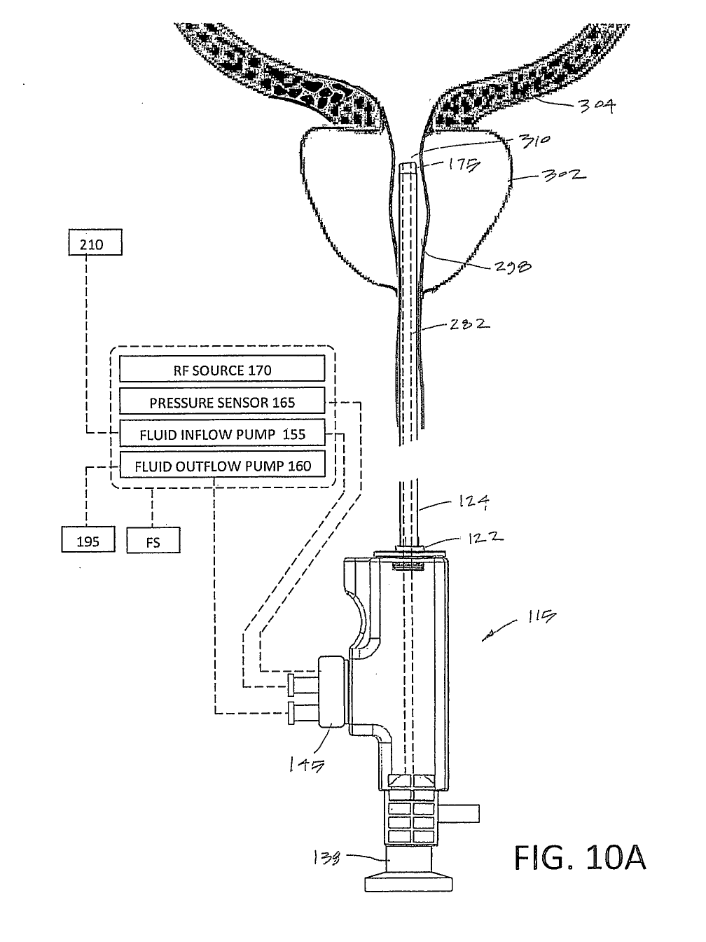

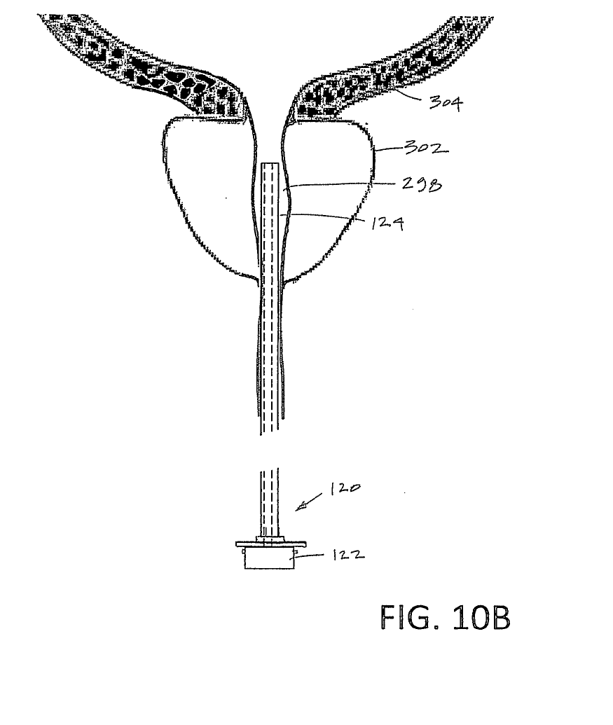

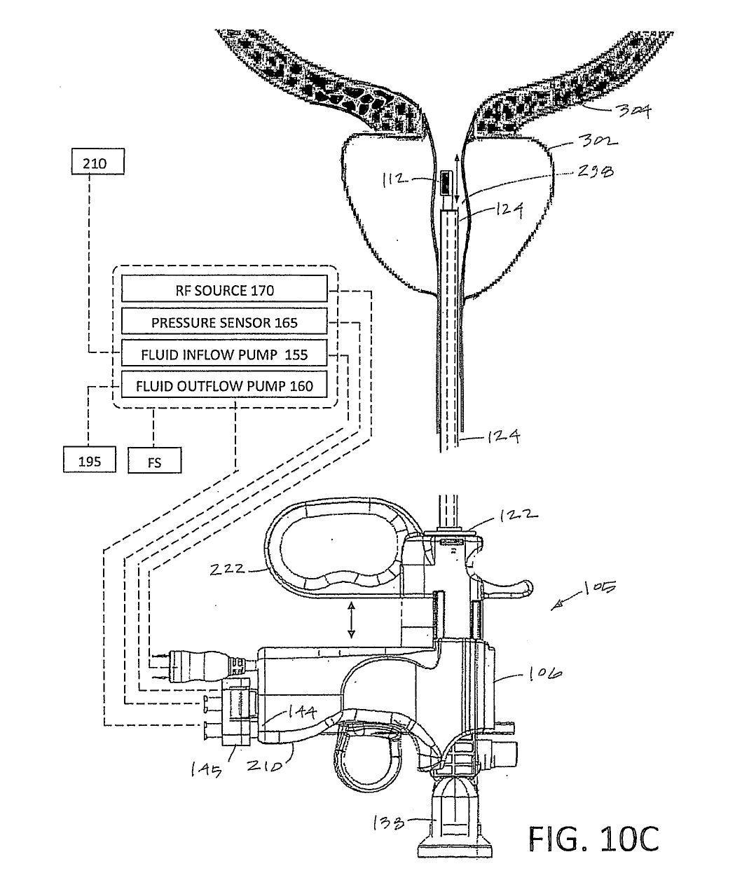

Now turning to FIGS. 10A-10C a method of using the system 100 of FIGS. 1 and 2 is shown. As a first step, the shaft 118 of the obturator device 115 it is inserted through through the sleeve 124 of introducer device 120. The hub 122 of introducer device 120 is locked into the handle 116 of the obturator 115. Thereafter tubing connector 145 is inserted into the fitting 144 in handle 116 of the obturator 115, which connects the inflow pump 155, outflow pump 160 and pressure sensor 165 to the obturator. The endoscope 138 is then inserted and locked into the interior channel of the obturator device 115. Thereafter, the controller 150 can be actuated by the physician to commence a fluid flow from source 210 and inflow source 155. Then, the physician can navigate the assembly shown in FIG. 10A through the patient's urethra 298 to access the prostate 302 under endoscopic vision together with the controlled irrigation. In one variation, the outflow pump 160 can be actuated simultaneously with the inflow pump 155 to circulate fluid prevent any fluid overpressure in the patient's urethra 298 and more particularly the bladder 304. In one aspect of the method, the inflow and outflow pumps 155, 160 can be preset to operate at flow rates ranging from 20 ml/min to 500 ml/min and more often from 50 to 200 ml/min. In another variation, the inflow pump 155 can be set to provide an inflow at a rate ranging from 20 ml/min to 500 ml/min and the outflow pump 160 can operate in response to fluid pressure in the working space 310 as measured by the pressure sensor 165. For example, the controller 150 can include an algorithm that activates or modulates the outflow pump 160 to maintain a set pressure in the working space of 20 mm Hg, 30 mm Hg, 40 mm Hg, 60 mm Hg, 80 mm Hg or any predetermined pressure therebetween.

FIG. 10B illustrates a subsequent step of the method wherein the obturator 115 is removed leaving the introducer device 120 in place. Thus, an access pathway is now provided for introduction of the resecting device 105 into the working space 310.

FIG. 10C illustrates a further step of the method wherein the endoscope 138 (after being removed from the obturator 115) is inserted into resecting device 105. Further, the connector 145 is inserted into fitting 144 in handle 106 of the resecting device 105, which the connects inflow pump 155, outflow pump 160, pressure sensor 165 and the RF source 170 to the resecting device 105. Thereafter, the assembly of the resecting device 105 and endoscope 138 is introduced through the introducer sleeve 124 to access the working space 310 in the prostate 290 or bladder 292. As can understood from FIG. 10C, the resecting device 105 then can be used by the physician as describe above to resect prostate tissue to treat BPH. More in particular, the resecting device 105 can be actuated by moveable grip 222 which is adapted to be squeezed toward stationary grip 220 to thus move the working end 112 axially back and forth. The physician can activate the electrosurgical function with a foot switch (FIG. 9) as described above to oscillate an energize the electrode 250 while reciprocating the working end 112 to resect tissue in a path. At the same time, the physician can rotate the shaft of the resecting device 105 so that the window surface 260 engages a wider path in the targeted tissue surface.

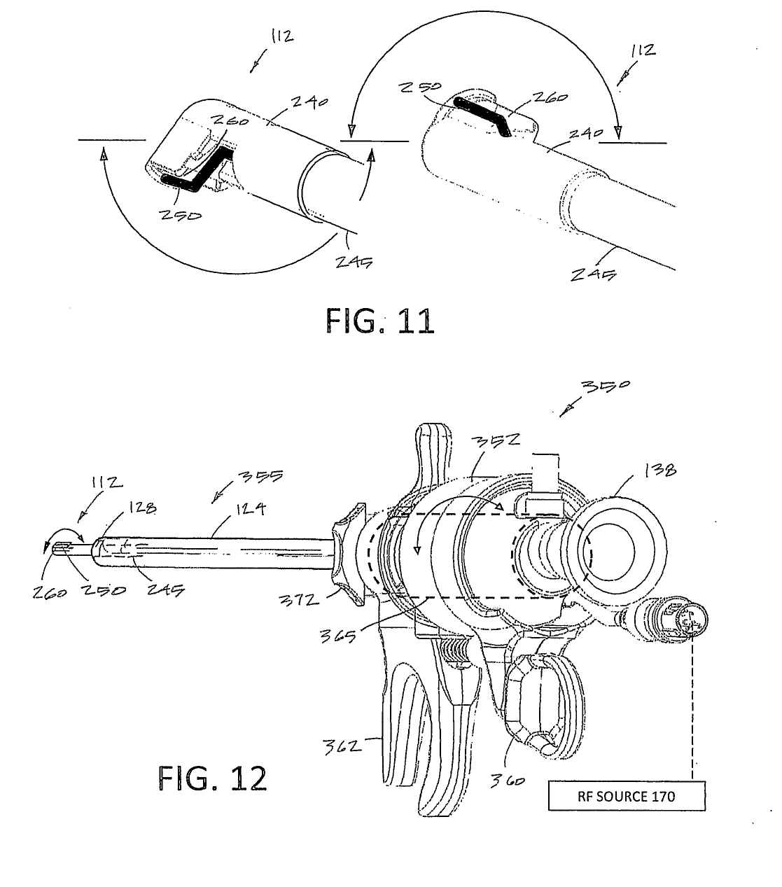

FIGS. 11 and 12 illustrates another variation of resecting device 350 that again has a handle 352 that is coupled to an extension portion 355 that carries a working end that can be the same working end 112 as described above in the previous embodiment. In FIG. 12, it can be seen that the handle 352 as a proximal stationary grip 360 and a distal movable grip 362 a can be used to reciprocate the working end 112 in the manner described previously. The resecting device 350 of FIG. 12 differs from the previous embodiment in that the handle 352 has a core portion 365 that is rotatable relative to the proximal and distal grips 360 and 362. The rotatable core 365 carries the endoscope 138 and thus allows physician to rotate the resecting device 350 and endoscope 138 by rotating the grip 372 and holding his or her hands in a stationary position which is convenient and comfortable during a resection procedure. FIG. 11 illustrates the working end 112 of the resecting device 350 be rotated into two different orientations. In one orientation, the window 260 and electrode 250 are facing downward and in the second orientation the window 260 and electrode 250 are facing in an upward direction as enabled by the rotatable core 365.

In another variation a second motor drive can be provided to reciprocate the working end while the first motor drive oscillates the electrode 250 across the window as described above. Such a variation can allow for manual movement of the grips to reciprocate the working end and also automated motor-driven reciprocation at one or more selected speeds, as well as a trigger to actuate a single reciprocation.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

What is claimed is:

1. A tissue resection system comprising:
an introducer sleeve with a hub;
an obturator having a shaft with a blunt tip, wherein the shaft and the blunt tip are adapted for insertion through the introducer sleeve; and
an electrosurgical resecting device with an elongated extension member adapted for insertion through the introducer sleeve.

2. The tissue resection system of claim 1, further comprising a controller console with at least a radiofrequency (RF) energy source, a fluid perfusion source, a fluid aspiration source, and a pressure sensor.

3. The tissue resection system of claim 2, wherein the obturator is configured to detachably connect to at least the fluid perfusion source, a fluid aspiration source, and a pressure sensor of the controller console.

4. The tissue resection system of claim 3, wherein the electrosurgical resecting device is configured to detachably connect to at least the fluid perfusion source, the fluid aspiration source, the pressure sensor, and the RF energy source of the controller console.

5. The tissue resection system of claim 4, further comprising an umbilical cable comprising at least tubes for connecting to the fluid perfusion source, the fluid aspiration source, and the pressure sensor.

6. The tissue resection system of claim 5, wherein the umbilical further comprised an RF cable for connecting to the RF source.

7. The tissue resection system of claim 5, further comprising an RF cable for connecting to the RF source, wherein the RF cable is separate from the umbilical.

8. The tissue resection system of claim 2, wherein the obturator shaft has first and second flow channels for fluidic communication with the fluid perfusion source and the fluid aspiration source respectively.

9. The tissue resection system of claim 2, wherein the extension member of the resecting device has first and second flow channels for fluidic communication with the fluid perfusion source and the fluid aspiration source respectively.

10. The tissue resection system of claim 8, wherein the obturator shaft further has an endoscope-receiving passageway extending therethrough.

11. The tissue resection system of claim 9, wherein the extension member of the resecting device further has an endoscope-receiving passageway extending therethrough.

12. The tissue resection system of claim 10, wherein the obturator shaft further has a third channel therethrough adapted for fluidic communication with a pressure sensor.

13. The tissue resection system of claim 11, wherein the extension member of the resecting device further has a third channel therethrough adapted for fluidic communication with a pressure sensor.

14. A method for treating a patient's prostate, said method comprising:
connecting an obturator to a controller console having a fluid perfusion source;
advancing a sleeve carrying the obturator and an endoscope though the patient's urethra while introducing a fluid from the controller console though the obturator into the urethra;
observing the urethra through the endoscope while advancing the sleeve and introducing the fluid;
ceasing to advance the sleeve after it is observed that a distal end of the sleeve has reached the prostate;
disconnecting the obturator from the controller console and removing the endoscope after ceasing to advance the sleeve;
exchanging the obturator for a resection device within the sleeve;
connecting the resection device to the fluid source and a radiofrequency (RF) energy source within the controller console;
placing the endoscope in the resection device; and
resecting the prostate with the resection device with RF energy from the RF energy source while circulating fluid from the controller console in the urethra and observing the resection with the endoscope.

15. A methods as in claim 14, wherein the controller console further includes at least a fluid aspiration source, and the method further comprises aspirating fluid from the urethra using the aspiration source while advancing the obturator and/or resecting the prostate.

16. A methods as in claim 15, wherein the controller console further includes at least a pressure sensor, and the method further comprises measuring pressure in the urethra using the pressure sensor while advancing the obturator and/or resecting the prostate.

17. A methods as in claim 16, wherein the fluid perfusion source, the fluid aspiration source, and the pressure sensor are interchangeably connected from the controller console to both the obturator and the resection device using a single umbilical cord.

18. A methods as in claim 17, further comprising wherein the RF energy source is individually connected from the controller console to the resection device using a power cord separate from the umbilical cord.
